(19) United States
(12) Patent Application Publication
(10) Pub. No.: US 2023/0181900 A1
(43) Pub. Date: Jun. 15, 2023

(54) Muscle Injury Prevention and Muscle Strengthening System

(71) Applicants: Avanti Ramraj, Granite bay, CA (US); SAMYUKTA SUDHEER, RANCHO CORDOVA, CA (US); Easha Narayanan, Roseville, CA (US); Keshav Shibu Sreedharan, Gold River, CA (US); MANI NARENDRA PANDIAN, FOLSOM, CA (US); Sai Amarthya Arjula, Rancho Cordova, CA (US); Diya Sivaramakrishnan Iyer, Folsom, CA (US)

(72) Inventors: Avanti Ramraj, Granite bay, CA (US); SAMYUKTA SUDHEER, RANCHO CORDOVA, CA (US); Easha Narayanan, Roseville, CA (US); Keshav Shibu Sreedharan, Gold River, CA (US); MANI NARENDRA PANDIAN, FOLSOM, CA (US); Sai Amarthya Arjula, Rancho Cordova, CA (US); Diya Sivaramakrishnan Iyer, Folsom, CA (US)

(21) Appl. No.: 17/550,394

(22) Filed: Dec. 14, 2021

(51) Int. Cl. A61N 1/36; A61N 1/04; A61N 1/02; A63B 71/06; G16H 20/40; G16H 20/30; G16H 10/60

(52) U.S. Cl. CPC A61N 1/36003; A61N 1/3603; A61N 1/0472; A61N 1/0452; A61N 1/025; A63B 71/0622; G16H 20/40; G16H 20/30; G16H 10/60; A63B 2071/0625; A63B 2225/50

(57) ABSTRACT

A muscle injury prevention and muscle strengthening system has an electrode harness, a controller, electrical muscle stimulation (EMS) electrodes, and an environmental sensor. The electrode harness secures around the patient's body holding the EMS electrodes in contact with the patient's body. The EMS electrodes send targeted electrical pulses at the motor nerves of the patient. The environmental sensor observes responses from the patient while monitoring external environmental factors. The environmental sensors range from EMG sensors to location sensors to gather important information about the patient and the environment. The controller manages the operation of the electrical components of the present invention. The present invention is fastened securely to the patient throughout all exercises and activities to properly stimulate the patient and gather responses from the patient.

### FIELD OF THE INVENTION

The present invention relates generally to an Electrical Muscle Stimulation and Electromyography sensor system designed to prevent muscle injury and atrophy while strengthening muscles for various individuals.

### BACKGROUND OF THE INVENTION

Within the United States around 8.6 million athletes become injured each year, with over half of these injuries being muscle related. Muscle atrophy is a common problem that affects a wide variety of individuals ranging from the elderly to adults who do not regularly exercise. The present invention intends to combine Electrical Muscle Stimulation (EMS), which sends electrical pulses to the motor nerves to exercise muscle fibers, and Electromyography (EMG), which monitors muscle contraction. This utilization of technology allows for the present invention to both prevent injury and strengthen muscles in various individuals. Along with the EMS and EMG technology the present invention utilizes machine learning to gather data and recommend customized EMS programs for each individual.

### BRIEF DESCRIPTION OF THE DRAWINGS

FIG. 1 is a front isometric perspective view of the present invention.

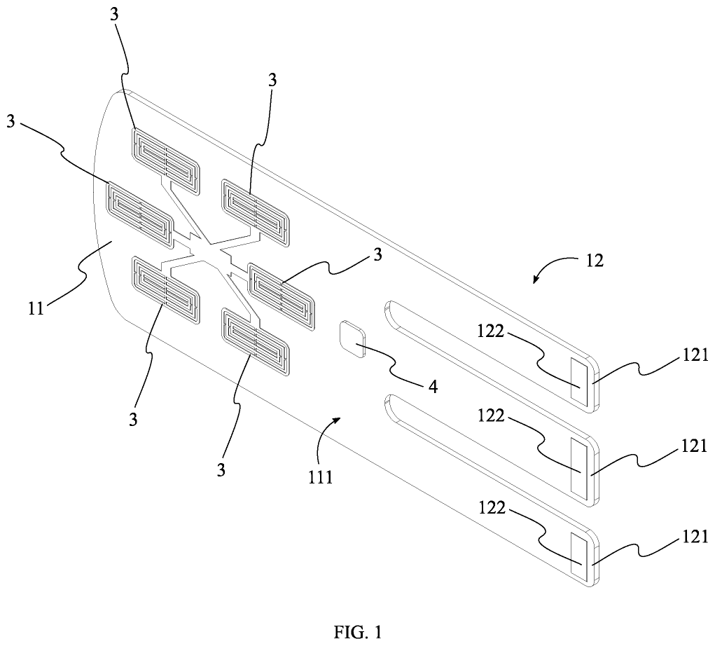

FIG. 2 is a rear isometric perspective view of the present invention.

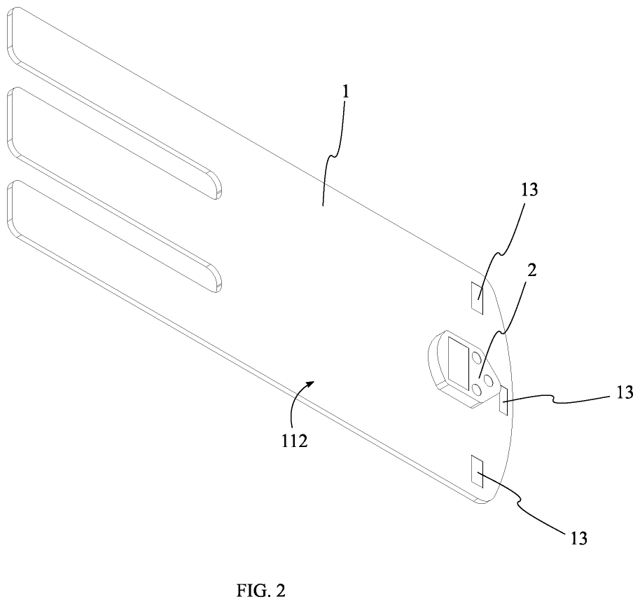

FIG. 3 is a front view of the present invention.

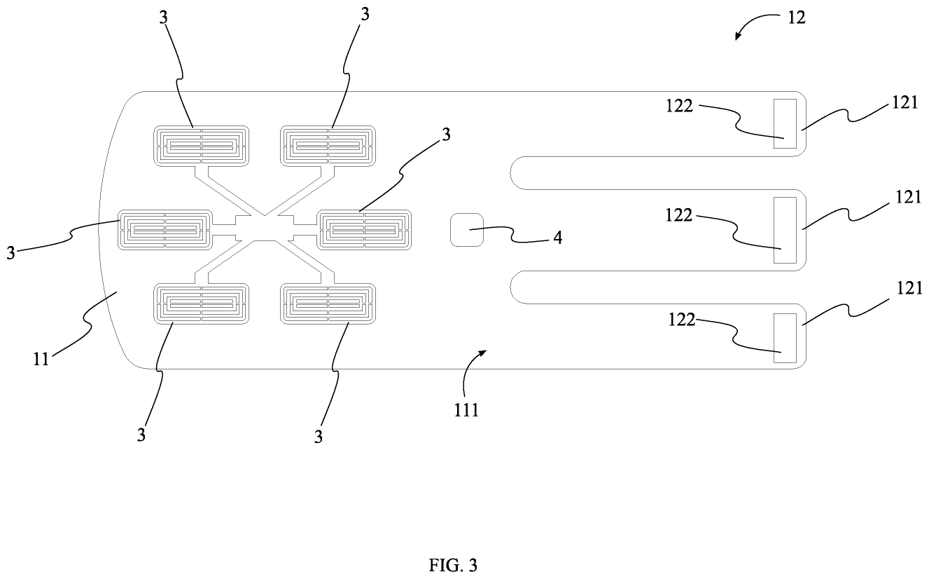

FIG. 4 is a rear view of the present invention.

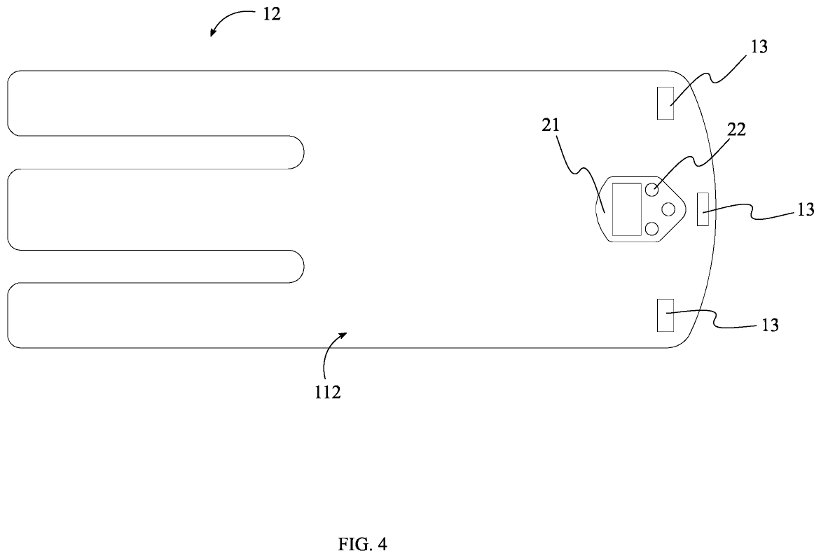

FIG. 5 is a right-side view of the present invention.

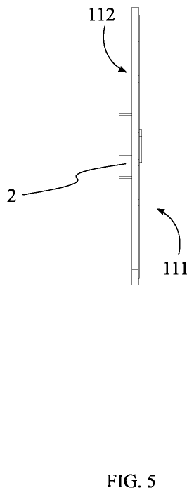

FIG. 6 is a left-side view of the present invention.

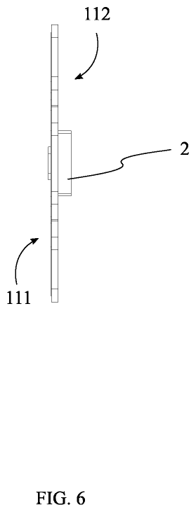

FIG. 7 is a top view of the present invention.

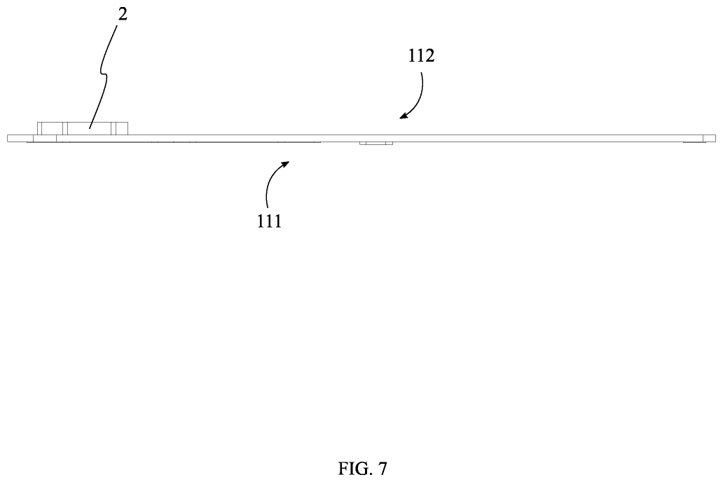

FIG. 8 is a bottom view of the present invention.

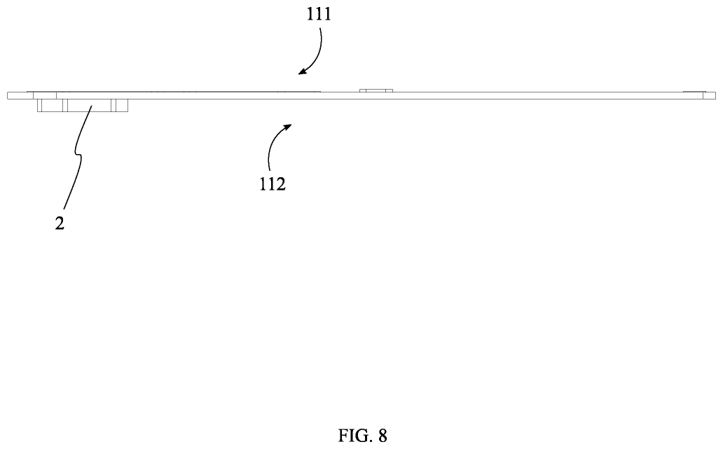

FIG. 9 is an illustration of the present invention secured to a patient's leg.

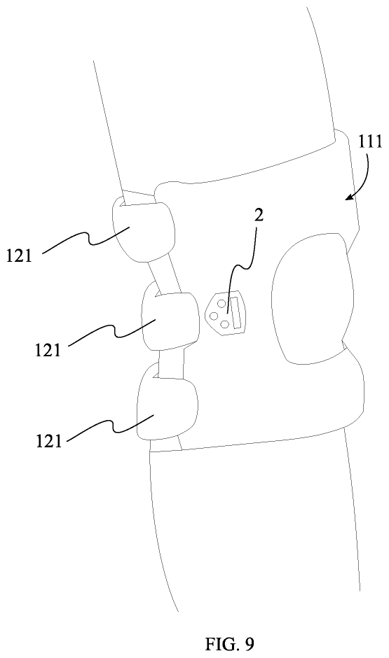

FIG. 10 is a block diagram of the electrode harness.

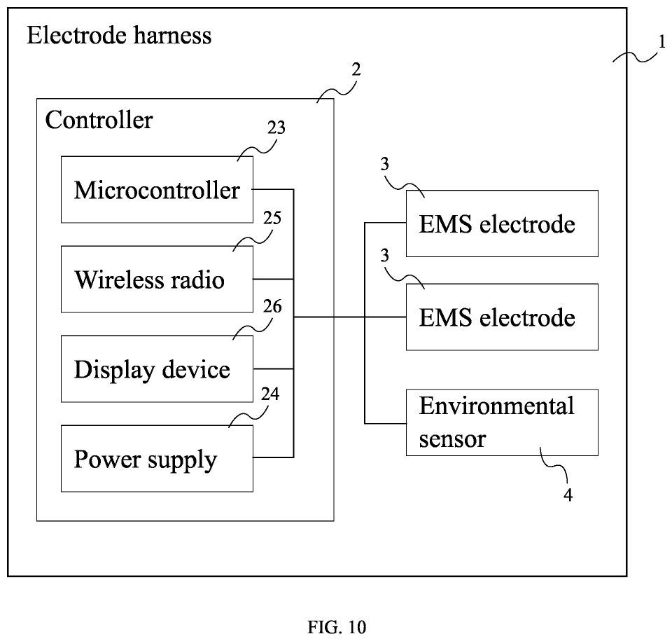

FIG. 11 is flow chart of the feedback loop.

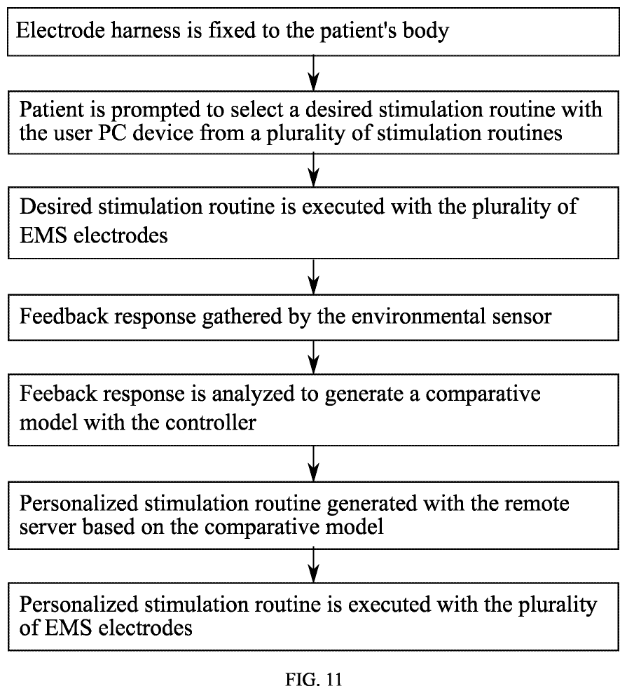

FIG. 12 is flow chart of the patient activity log.

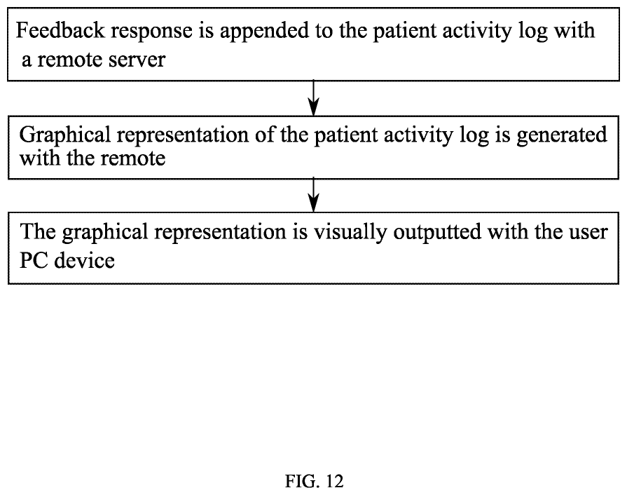

FIG. 13 is flow chart of the analyzation of the activity log.

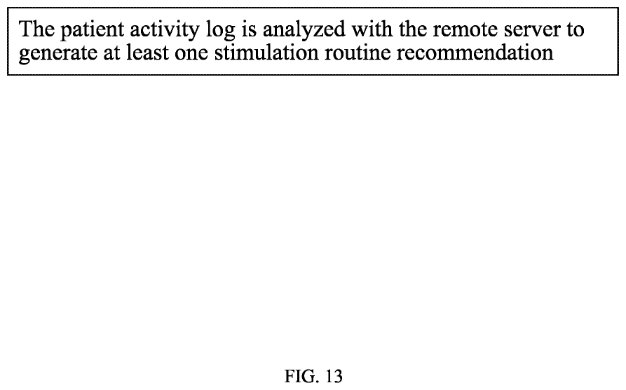

FIG. 14 is flow chart of the professional recommendation.

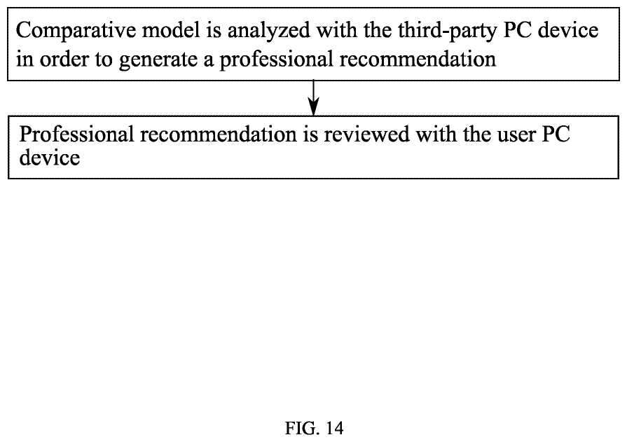

FIG. 15 is flow chart of the virtual user model.

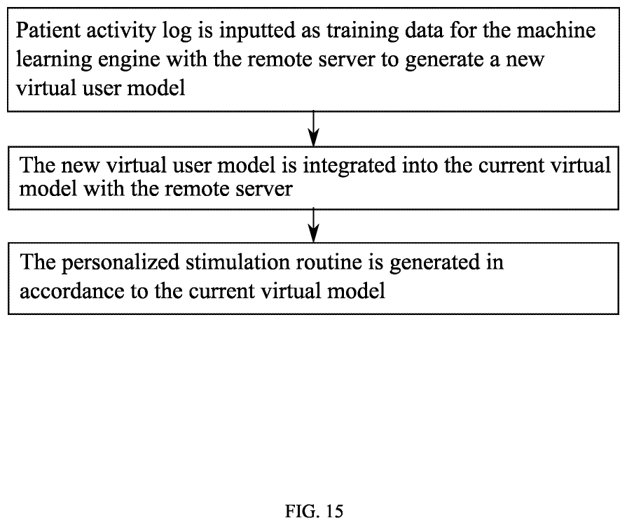

### DETAIL DESCRIPTIONS OF THE INVENTION

All illustrations of the drawings are for the purpose of describing selected versions of the present invention and are not intended to limit the scope of the present invention.

The preferred embodiment of the present invention comprises at least one electrode harness 1, at least one controller 2, a plurality of electrical muscle stimulation (EMS) electrodes 3, and at least one environmental sensor 4. The electrode harness 1 is a flexible brace that is able to be affixed to a patient's body. The controller 2 is an electronic system intended to manage the operation of the electrical components of the present invention. The plurality of EMS electrodes 3 is a group of electrical stimulators intended to send electrical pulses targeted at the motor nerves of the patient. The environmental sensor 4 is an electronic device designed to gather feedback responses from the patient and to monitor the state of the patient's body and the external environment. Specifically, embodiments of the present invention include at least one environmental sensor 4 selected from the group comprising EMG sensors, accelerometers, temperature sensors, pulse oximeters, location sensors, touch sensors, and moisture sensors. The electrode harness 1 comprises a main body 11 and at least one patient-mounting device 12. The patient-mounting device 12 is a fastening mechanism used to detachably couple the electrode harness 1 to the patient's body. As such, the electrode harness 1 is able to be wrapped around the desired body part of the patient, ensuring a secure fit throughout any continuous movement from the patient while the electrode harness 1 is attached. The plurality of EMS electrodes 3 is integrated into a patient-facing surface 111 of the main body 11. As a result, the EMS electrodes is able to contact the user's skin and deliver electrical stimulation. This enables the present invention to be applied to various sections of the user's body for rehabilitation and strength training. The plurality of EMS electrodes 3 is distributed across the patient-facing surface 111 of the main body 11. Consequently, the plurality of EMS electrodes 3 is able to make contact with the patient's skin in a variety of specified locations, ensuring the EMS electrodes is able to target at least one motor nerve or muscle group. The environmental sensor 4 is integrated into the patient-facing surface 111 of the main body 11. Thus, the environmental sensor 4 is able to monitor the muscle feedback in response to electrical stimulation from the plurality of EMS electrodes 3. Further, the environmental sensor 4 is able to gather information about other aspects of the patient's physiological state and the external environment. The controller 2 is mounted adjacent to a provider-facing surface 112 of the main body 11, opposite to the environmental sensor 4. So, the controller 2 is able to receive input controls from the patient or provider and display information to the patient or provider. Additionally, this configuration enables the patient-facing surface 111 of the present invention to be focused on analyzing the patent's physiology while the provider-facing surface 112 acts as an interface for information display and user input. The plurality of EMS electrodes 3 and the environmental sensor 4 are electronically connected to the controller 2. As a result, the controller 2 is able to control the electrical stimulation output of the plurality of EMS electrodes 3 and monitor the stimulated muscle response. The patient-mounting device 12 is connected adjacent to the main body 11. Consequently, the patient-mounting device 12 is able to wrap around the patient and create a loop that is tethered to the opposite side of the main body 11. The patient-mounting device 12 is positioned offset from the plurality of EMS electrodes 3 across the main body 11. Thus, the patient-mounting device 12 is able to secure the present invention to the patient without interfering with the electrical signal output from the plurality of EMS electrodes 3.

Furthermore, the muscle injury prevention and muscle strengthening system as described in the previous paragraph also comprises at least one anchor point 13. The anchor point 13 is an area on the main body 11 to which the patient-mounting device 12 is able to be attached. Preferably, the anchor point 13 and the patient-mounting device 12 employ a hook and loop fastener method or in alternative embodiments quick-release fasteners, buttons, clasps, buckles and the like. The patient-mounting device 12 comprises at least one strap 121 and at least one fastening device 122. As a result, the patient-mounting device 12 is able to wrap around the patient with the strap 121 and attach to the anchor point 13 with the fastening device 122 wherein the fastening device 122 is a coupling device that is detachably coupled to a corresponding anchor point 13. The anchor point 13 is mounted adjacent to the provider-facing surface 112. Consequently, the anchor point 13 positioning allows for easy access to the patient or provider to secure the patient-mounting device 12. The strap 121 is terminally connected to the main body 11. Thus, the strap 121 creates a snug fit around the patient when wrapped, to ensure the present invention stays properly secured when being used. The fastening device 122 is terminally connected to the strap 121, opposite to the main body 11. So, the fastening device 122 creates a range of attachment sizes to ensure the muscle injury prevention and muscle strengthening system is able to accommodate and be secured and function properly around various patients with different body sizes and shapes. The fastening device 122 is detachably connected to the anchor point 13. As a result, the fastening device 122 is able to easily be secured and removed from the anchor point 13 making a convenient assembly around the patient. In some embodiments, a plurality of straps 121, fastening devices 122, and anchor points 13 to provide a multipoint harness that is able to be adjusted around the patient's body, as required.

In some embodiments, the fastening device 122 is mounted onto the patient-facing surface 111. Consequently, the configuration of the anchor point 13 and fasting device allows for easy access to the patient or provider to secure the patient-mounting device 12.

Additionally, the present invention comprises a controller 2, a control panel 22, a microcontroller 23, and a power supply 24. The controller 2 is an electronic device that further comprises a housing 21, a control panel 22, a microcontroller 23 and a power supply 24. The housing 21 is a hollow shell affixed to the main body 11 designed to hold various operational components of the present invention. The control panel 22 is an electronic device that is able to receive physical input from the patient or provider and output relevant information. The microcontroller 23 is an electronic device that translates the physical inputs to a computer command and executes said command. The power supply 24 is an electrical storage unit designed to power various components within the controller 2. The control panel 22 is integrated into a lateral sidewall of the housing 21. As a result, the control panel 22 is able to be interacted with by the patient or provider, which sends commands to the microcontroller 23. The microcontroller 23 is mounted within the housing 21. Consequently, the microcontroller 23 is protected from external elements that could interfere with the signals being send and received by the microcontroller 23. The power supply 24 is mounted within the housing 21. Thus, the power supply 24 is able to be easily connected to the microcontroller 23 and control panel 22 to provide the necessary power needed for each component to operate correctly. The control panel 22, the power supply 24, the plurality of EMS electrodes 3, and the environmental sensor 4 are all electronically connected to the microcontroller 23. So, the control panel 22, the power supply 24, the plurality of EMS electrode 3, and the environmental sensor 4 is able to all send and receive electronic signals with the microcontroller 23, creating an interconnected circuit between all components.

The present invention is designed to be capable of remote control and communication. The controller 2 further comprises a wireless radio 25. As a result, the controller 2 is capable of remote communication and control, adding more functionality to the controller 2 than the control panel 22 inputs. The wireless radio 25 is mounted within the housing 21. Consequently, this positions the wireless radio 25 in close proximity to the power supply 24 and wireless radio 25, allowing for both electric and electronic connections respectively. The wireless radio 25 is electronically connected to the microcontroller 23. Thus, the wireless radio 25 is able to transfer data between the microcontroller 23 to a remote computing device.

The muscle injury prevention and muscle strengthening system provides feedback to the patient or provider via the controller 2. The controller 2 further comprises a display device 26. The display device 26 is an electronic screen that indicates various information to the patient or provider such as the selected mode and the electrical pulse EMS electrode output level. The display device 26 is mounted onto the lateral sidewall of the housing 21. As a result, the display device 26 is able to be easily viewed by the patient or provider. The display device 26 is positioned offset from the control panel 22. Consequently, the display device 26 is able to be viewed and is not obstructed when the patient or provider is interacting with the control panel 22. The display device 26 is electronically connected to the microcontroller 23. Thus, the display device 26 is able to display information relayed from the microcontroller 23.

The present invention also pertains to a method of employing the muscle injury prevention and muscle strengthening system. The system for executing the method of the present invention provides a plurality of stimulation routines stored on at least one remote server. Each stimulation routine refers to a subprocess that is executed to direct the plurality of EMS electrodes 3 to output specific electrical frequencies for specific time intervals. Additionally, each of the plurality of EMS electrodes 3 may be directed to output a unique electrical frequency so that the patient is subjected to multiple frequencies, simultaneously. The term “remote server” is used herein to refer to a computing device capable of executing all background processes required to perform the method of the present invention. The method of the present invention utilizes machine learning to analyze historical sensor data and recommend a desired stimulation routine to the patient. Additionally, the method of the present invention provides at least one user profile that is associated to at least one user personal computing (PC) device and managed by the remote server. The term “computing device” is used herein to refer to any electronic system capable of executing the method of the present invention and communicating with external devices. In some embodiments, the user employs computing devices selected from the group including, but not limited to, smart phones, smart glasses, tablet computers, and laptop computers. The user profile is a virtual representation of the user and includes user preferences, as well as user identification data. In its preferred embodiment the present invention comprises at least one electrode harness 1, at least one controller 2, a plurality of EMS electrodes 3, and at least one environmental sensor 4. The electrode harness 1 is first affixed to the patient's body. As a result, the electrode harness 1 is placed in contact with a desired location upon the patient. The method continues by prompting the patient or provider to select a desired stimulation routine with the user PC device, wherein the desired routine is from the plurality of stimulation routines. Consequently, the stimulation routine is customized to fit the needs and body type of the patient to provide optimal stimulation and feedback. The method continues by executing the desired stimulation routine with the plurality of EMS electrodes 3. Thus, the stimulation routine directs the microcontroller 23 to elicit a desired set of electrical pulses from the plurality of EMS electrodes 3. These electrical pulses are targeted at the motor nerves within the desired body part of the patient. Furthermore, a feedback response is gathered with the environmental sensor 4. So, the environmental sensor 4 gathers data that monitors how the patient reacts to the stimulation routine. Thereby measuring the effectiveness of the stimulation profile and indicating any fatigue within the patient muscle. Afterwards, the feedback response is utilized in order to generate a comparative model with the controller 2. The comparative model refers to sample data built by machine learning algorithms based on the feedback responses that are automatically updated when additional data is gathered. As a result, the comparative model is able to identify deficiencies on the treatment and to develop unique stimulation routines to address these deficiencies. The method of the present invention continues when a personalized stimulation routine is generated with the remote server based on the comparative model. The personalized stimulation routine refers to a customized activation sequence of the plurality of EMS electrodes 3 based on the patient needs and preferences. Consequently, the comparative model ensures that the stimulation routine is optimally designed for the patient and their desired result. Finally, the personalized stimulation routine is executed with the plurality of EMS electrodes 3. Thus, the plurality of EMS electrodes 3 sends electric pulses to the motor nerves of the patient based on the stimulation routine that was optimally designed through machine learning.

The user profile is associated to at least one user PC device. The user profile includes at least one patient activity log. The patient activity log contains records of the feedback responses of the patient gathered by the environmental sensor 4 after each use of the present invention. Furthermore, the patient activity log is able to contain previous stimulation routines completed by the patient, the elapsed time since the last completed stimulation routine, the data from previous activities, and the rehabilitation progress of the patient based off the user feedback to the stimulation routines over a set period of time. The patient activity log is then appended with the current feedback response with the remote server. As a result, the remote server is updated with new data each time the patient selects a stimulation routine, and a feedback response is gathered. Further, a graphical representation of the patient activity log is generated with the remote server. Consequently, the patient activity log is able to be displayed in a numbered or graphical format to enable the patient or provider to show patient progress compared to a variable such as time in a visually appealing manner. Finally, the graphical representation is visually outputted with the user PC device. Thus, the user PC device allows the comparative model to be seen in a more detailed graphical representation.

The method of the present invention utilizes machine learning to optimally provide the best experience to the patient. The machine learning achieves this by completing a series of steps by taking the patient records and comparing them to various generalized stimulation routines, subjecting the patient to the best possible generalized stimulation routine, recording and analyzing the patient feedback, comparing the analyzed feedback to the stimulation metrics to find a better stimulation routine, executing the better stimulation routine, recording patient feedback from the better stimulation routine, and looping back to the first step to repeat the process. The patient activity log is analyzed with the remote server in order to generate at least one stimulation routine recommendation. As a result, the stimulation routine recommendation is tailored specifically to the patient due to their previous stimulation routine response and desired result. This enables the present invention to constantly improve over time as more feedback data is stored within the remote server relating to the user profile.

The method of the present invention allows the feedback data to be remotely sent to alternative devices. The present invention provides at least one third-party profile managed by the remote server, wherein the profile is associated to at least one third-party PC device. The third-party profile is able to be associated with any individual such as a health care provider, family member, etc. allowing the data stored by the present invention to be remotely shared. The comparative model is analyzed with the third-party PC device in order to generate a professional recommendation. Thus, the third-party PC device ensures that a professional recommended stimulation routine is able to be provided even without the individual being within the vicinity of the present invention. Next the patient is prompted to review professional recommendation with the user PC device. So, the PC device will allow the patient to take the professional recommendation into consideration when selecting a stimulation routine.

The muscle injury prevention and muscle strengthening system constantly learning and improving based on the reactions observed about the patient and the professional recommendations. The present invention provides a current virtual user model is included in the user profile and provides a machine learning engine managed by the remote server. The virtual model is a combination of previously recorded data associated with the feedback responses of the patient during each stimulation routine. The machine learning engine is an algorithm that utilizes the various inputs such as the virtual model and the professional recommendations to update a new virtual model and output a stimulation routine recommendation. The patient activity log inputs training data for the machine learning engine with the remote server in order to generate a new virtual user model. As a result, the virtual user model is constantly being updated after each stimulation routine is complete and feedback response data is gathered from the patient. Further, the new virtual user model is integrated into the current virtual model with the remote server. Consequently, the virtual model is able to retain a history of data relating to the feedback response of the patient to the stimulation routine for use in displaying progress over a period. Finally, the personalized stimulation routine is generated in accordance with the current virtual model. Thus, the virtual model is constantly updated allowing an optimal stimulation routine to be provided to the patient that is unique given their desired routine, feedback response history, physical parameters, and professional recommendation.

Although the invention has been explained in relation to its preferred embodiment, it is to be understood that many other possible modifications and variations can be made without departing from the spirit and scope of the invention as hereinafter claimed.

What is claimed is:

1. A muscle injury prevention and muscle strengthening system comprising:
at least one electrode harness;
at least one controller;
a plurality of electrical muscle stimulation (EMS) electrodes;
at least one environmental sensor;
the electrode harness comprising main body and at least one patient-mounting device;
the plurality of EMS electrodes being integrated into a patient-facing surface of the main body;
the plurality of EMS electrodes being distributed across the patient-facing surface of the main body;
the environmental sensor being integrated into the patient-facing surface of the main body;
the controller being mounted adjacent to a provider-facing surface of the main body, opposite to the environmental sensor;
the plurality of EMS electrodes and the environmental sensor being electronically connected to the controller;
the patient-mounting device being connected adjacent to the main body; and
the patient-mounting device being positioned offset from the plurality of electrodes across the main body.

2. The muscle injury prevention and muscle strengthening system as claimed in claim 1 comprising:
at least one anchor point;
the patient-mounting device comprising at least one strap and at least one fastening device;
the anchor point being mounted adjacent to the provider-facing surface;
the strap being terminally connected to the main body;
the fastening device being terminally connected to the strap, opposite to the main body; and
the fastening device being detachably connected to the anchor point.

3. The muscle injury prevention and muscle strengthening system as claimed in claim 2, wherein the fastening device being mounted onto the patient-facing surface.

4. The muscle injury prevention and muscle strengthening system as claimed in claim 1 comprising:
the controller comprising a housing, a control panel, a microcontroller, and a power supply;
the control panel being integrated into a lateral sidewall of the housing;
the microcontroller being mounted within the housing;
the power supply being mounted within the housing; and
the control panel, the power supply, the plurality of EMS electrodes, and the environmental sensor being electronically connected to the microcontroller.

5. The muscle injury prevention and muscle strengthening system as claimed in claim 4 comprising:
the controller further comprising a wireless radio;
the wireless radio being mounted within the housing; and
the wireless radio being electronically connected to the microcontroller.

6. The muscle injury prevention and muscle strengthening system as claimed in claim 4 comprising:
the controller further comprising a display device;
the display device being mounted onto the lateral sidewall of the housing;
the display device being positioned offset from the control panel; and
the display device being electronically connected to the microcontroller.

7. The method for operating a muscle injury prevention and muscle strengthening system comprising:
providing a plurality of stimulation routines stored on at least one remote server;
providing at least one user profile managed by the remote server, wherein the user profile is associated to at least one user personal computing (PC) device;
at least one electrode harness;
at least one controller;
a plurality of electrical muscle stimulation (EMS) electrodes;
at least one environmental sensor;
affixing the electrode harness to a patient's body;
prompting to select a desired stimulation routine with the user PC device, wherein the desired routine is from the plurality of stimulation routines;
executing the desired stimulation routine with the plurality of EMS electrodes;
gathering a feedback response with the environmental sensor;
analyzing the feedback response in order to generate a comparative model with the controller;
generating a personalized stimulation routine with the remote server based on the comparative model; and
executing the personalized stimulation routine with the plurality of EMS electrodes.

8. The method as claimed in claim 7 comprising:
providing the user profile includes at least one patient activity log;
appending the feedback response to the patient activity log with the remote server;
generating a graphical representation of the patient activity log with the remote server; and
visually outputting the graphical representation with the user PC device.

9. The method as claimed in claim 8 comprising:
analyzing the patient activity log with the remote server in order to generate at least one stimulation routine recommendation.

10. The method as claimed in claim 7 comprising:
providing at least one third-party profile managed by the remote server, wherein the profile is associated to at least one third-party PC device;
analyzing the comparative model with the third-party PC device in order to generate a professional recommendation; and
prompting to review professional recommendation with the user PC device.

11. The method as claimed in claim 7 comprising:
providing a current virtual user model is included in the user profile;
providing a machine learning engine managed by the remote server;
inputting the patient activity log as training data for the machine learning engine with the remote server in order to generate a new virtual user model;
integrating the new virtual user model into the current virtual model with the remote server; and
generating the personalized stimulation routine in accordance to the current virtual model.
